(19) United States
(12) Patent Application Publication
(10) Pub. No.: US 2008/0295774 A1
(43) Pub. Date: Dec. 4, 2008

(54) Automated Packaging, Inoculation, and Harvesting of Lepidopterous Larvae for Protein Manufacturing

(75) Inventors: Nikolai A. Van Beek, Northeast, MD (US); David C. Davis, Newark, DE (US); Thomas A. Curran, Wilmington, DE (US); Wayne C. Taylor, Newark, DE (US)

Correspondence Address: VENABLE LLP, P.O. BOX 34385, WASHINGTON, DC, 20043-9998, US

(73) Assignee: Chesapeake Perl, Inc., Savage, MD (US)

(21) Appl. No.: 11/884,029

(22) PCT Filed: Feb. 10, 2006
(86) PCT No.: PCT/US2006/004543

§ 371 Date: Aug. 8, 2008

Related U.S. Application Data
(60) Provisional application No. 60/651,629, filed on Feb. 11, 2005.

(51) Int. Cl. A01K 67/033

(52) U.S. Cl. 119/6.6

(57) ABSTRACT

A method for mass rearing of insects. A web including a plurality of wells is provided. Insect eggs or larvae and growth medium are loaded in the wells. The wells are covered. The web is rolled up. The eggs or larvae are incubated during eggs hatch and larval growth. The web is unrolled and the larvae harvested.

### FIELD OF THE INVENTION

The invention relates to a method and a system for mass rearing of insects. More particularly, the general field of this invention relates to the automation of the process of growing large numbers of larvae that are healthy and synchronous in their development, an optionally to inoculate these larvae with a recombinant baculovirus, and to harvest the infected larvae.

### BACKGROUND OF THE INVENTION

Many lepidopterous insect larvae are agricultural pest insects and methods have been developed to rear large numbers of certain insects for screening/assaying of potential insecticides or for sterile insect release programs. In addition, the larvae of certain lepidopterous species are being used to produce large quantities of baculovirus as the active ingredient for commercially available bio-insecticides. Finally, baculoviruses when genetically engineered can express large amounts of valuable proteins in lepidopterous larvae.

The life cycle of lepidopterous insects consists of 4 stages (egg, larva, pupa, and adult). The larval stage is the target stage for above uses, except for the sterile release programs where usually pupae/adult insects are the target stage.

Currently utilized processes for mass rearing of insects typically utilize a so-called form, fill, and seal (FFS) machine. The machine, in a continuous process, thermoforms indentations, or wells, in a film of PVC typically referred to as a web. The machine then fills the wells with liquid insect diet. The insect diet may also be extruded into the thermoformed wells. After filling the wells with diet, the diet is cooled until it solidifies. The machine then deposits insect eggs or neonate larvae in the wells. The machine then covers the wells with perforated lidding material to form an enclosed insect habitat and cuts the web in sections that can be handled manually. This ends the continuous process, and further handling is carried out in batches

This process typically operates at a speed of 5-10 indexes per minute. Each index may produce habitats for up to several hundreds of larvae either in one large habitat or many small habitats. During this process a several hundred feet-long film of PVC may be transformed into discrete trays, filled with either insect larvae or insect eggs. These trays are usually stacked on carts and placed in an incubation room. Then, optionally after several days of incubation when larvae have reached the appropriate size, they undergo the step of viral inoculation. In some cases, the larvae are not inoculated and may remain in the wells to develop into adult insects.

Insects that are being used to produce virus or an exogenous protein product are inoculated. During the inoculation step the trays are typically handled manually, as they are removed from the cart and inserted one-by-one in the inoculator. After inoculum has been sprayed on the diet the trays are manually stacked back onto a cart. After inoculation the larvae are again incubated for some days, and finally the trays are taken from the carts manually, one-by-one, for larval harvest.

The techniques described above are very labor intensive. The output of the form, fill, and seal machine is in the form of relatively small, discrete habitat trays. These units must be frequently handled by operators in subsequent steps in the process.

### SUMMARY OF THE INVENTION

The present invention provides a method for mass rearing of insects. A web including a plurality of wells is provided. Insect eggs and growth medium are loaded in the wells. The wells are covered. The web is rolled up. The eggs and larvae after the eggs hatch are incubated. The web is unrolled. The larvae are harvested.

The invention also relates to a system for mass rearing of insects, including a web including a plurality of wells operative to receive insect eggs and growth medium and a first spool operative to roll up the web after loading the wells with insect eggs and growth medium, to store the web at least during incubation of the eggs and larvae after hatching of the eggs, and to unroll the web to harvest the larvae.

The invention also relates to a structure for mass rearing of insects, including a web having a length of about 50 feet to about 400 feet, a matrix of wells arranged in the web, growth medium and insect larvae arranged in the wells, and a cover covering the wells.

The invention further relates to a harvester for harvesting insect larvae from a web of wells. The harvester includes a web unwinder operative to unwind the web from a spool on which it is wound, a top film remover operative to remove the top film from the web of wells and a separator operative to isolate insect larvae removed from the wells.

The invention also includes an inoculator for mass inoculation of insect larvae with virus. The inoculator includes a web unwinder operative to unwind a web of wells, each well containing at least one insect larvae, a web support and guide operative to support and guide the web during inoculation, a matrix of injection needles operative to inject inoculum into the wells, an inoculum manifold operative to deliver inoculum to the matrix of needles, and at least one motor operative to alter the location of at least one of the web and the matrix of injection needles after injecting inoculum in the wells.

### BRIEF DESCRIPTION OF THE DRAWINGS

Objects and advantages of the present invention will be more clearly understood from the following specification when considered in conjunction with the accompanying drawings, in which:

FIG. 1a is a flowchart that illustrates a process overview for an embodiment of a process of protein manufacturing in insect larvae, including egg production, virus production, and infected larva production;

FIG. 1b is a flowchart that illustrates aspects of an embodiment of insect rearing, infection, and harvest;

FIG. 2 represents a photograph showing part of an embodiment of a web with insect larvae occupying diet-filled wells;

FIG. 3a represents a side view of an embodiment of a spool that may be utilized to roll up, store and unroll as web;

FIG. 3b represents an end view of the embodiment of the spool shown in FIG. 3a;

FIG. 3c depicts an embodiment of an empty spool;

FIG. 4a is a side view of an inoculator for inoculating insects with virus;

FIG. 4b is a scale image of two 4×7 inoculator arrays over a feed rack;

FIG. 5 is a schematic image of an embodiment of a harvester for harvesting larvae;

FIG. 6A is a side view of another embodiment of a spool according to the present invention; and

FIG. 6B is a side view of the spool shown in FIG. 6A about which a web has been rolled.

### DETAILED DESCRIPTION OF PREFERRED EMBODIMENTS

Embodiments of the present invention provide a significantly more efficient process as compared to currently utilized manual, batch processes. The present invention can reduce labor by about 40% to about 60% and by as much as about 80%. The reduction in labor requirements flows at least in part from the possibility to automate certain aspects of the present invention. The present invention can also eliminate some steps required according to presently utilized processes. As a result, the present invention can reduce operator time and the need for multiple operators.

A number of purposes exist for mass rearing of insects. One reason is to provide test insects for screening/assaying insecticidal compounds. Another reason is to manufacture bioinsecticides such as recombinant or wild-type baculoviruses, entomopox viruses, entomopathogenic fungi, or nematodes, for example. Additionally, insects may be mass reared to manufacture recombinant proteins using baculovirus-mediated expression. Furthermore, insects may be mass reared for use in sterile insect release programs.

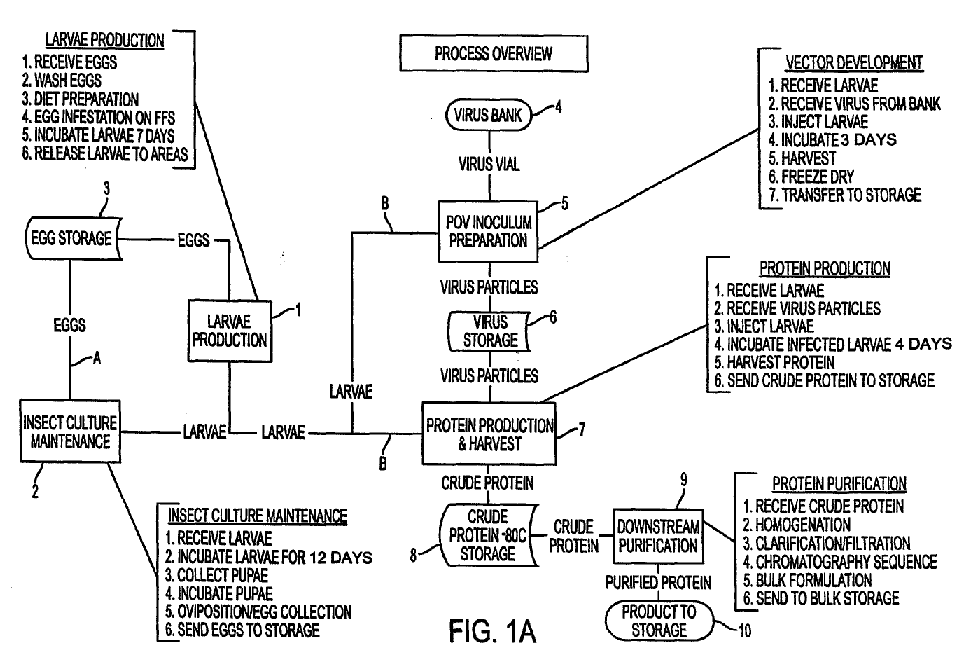

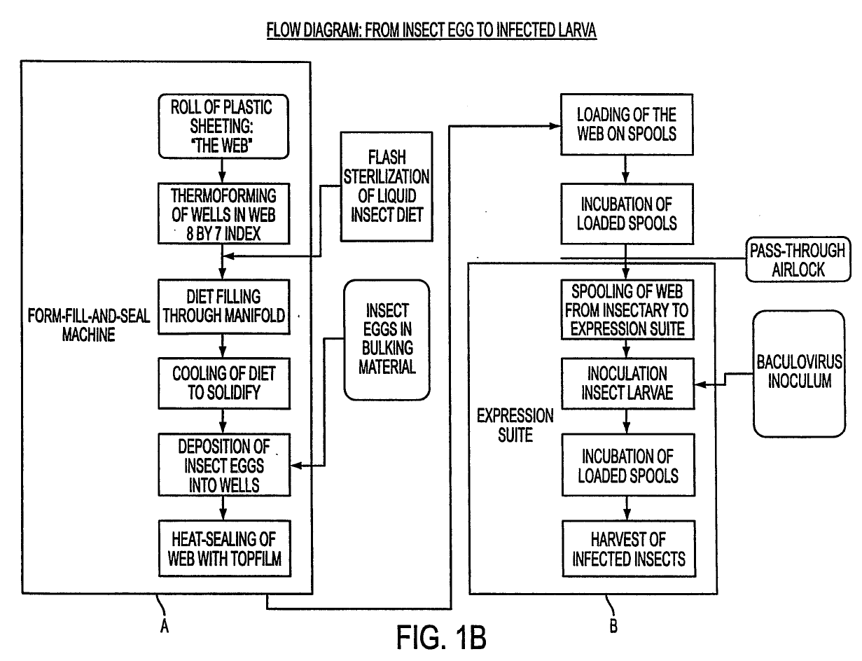

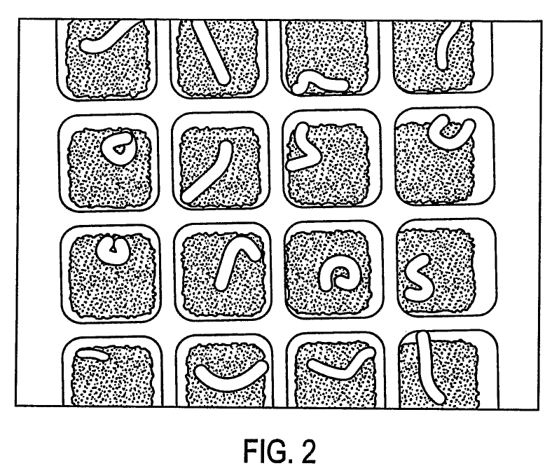

FIG. 1 is a flowchart that illustrates an overview of a process for larva-mediated protein manufacturing. Insect eggs storage 3 provides eggs for larvae production 1 and insect culture maintenance 2. According to this process, larvae production includes receiving the eggs, washing the eggs, preparing the diet, delivering the eggs to a web of wells with an FFS machine, incubating the eggs and releasing the larvae. In this case, the eggs are incubated for seven days.

Insect culture maintenance, which maintains a supply of eggs, can include receiving larvae from the larvae production process. The larvae are incubated for twelve days and the resulting pupae collected. The pupae are incubated and oviposition/egg collection carried out. Eggs are then sent to storage as indicated by pathway A.

Larvae may also be utilized in POV inoculum preparation 5, as indicated by pathway B. Inoculum preparation includes receiving the larvae and receiving the virus from virus bank 4. The larvae are injected with the virus and incubated for three days. The virus is harvested, freeze dried and transferred to virus storage 6.

Rather than being utilized to manufacture virus, the larvae may be utilized for protein production and harvest 7. As such, the larvae may be obtained from larvae production 1 as indicated by pathway C. Larvae and virus particles are received and the larvae inoculated with the recombinant virus. The larvae for protein production may be infected orally with POV. The resulting infected larvae may be incubated for four days. The infected larvae may then be harvested and the crude protein sent to storage.

Infected larvae storage 8 may take place in conditions of −80° C. Infected larvae may be transferred to downstream purification 9. The infected larvae received from storage are homogenized, clarified and filtered. A sequence of chromatography or other protein purification protocol is carried out. Bulk protein formulation is derived and sent to bulk storage 10.

FIG. 1b illustrates a flowchart that illustrates elements of a process for producing infected larvae. This process may be divided into two general portions. The first portion A includes steps leading up to producing a web of wells loaded with larvae. To being, a roll of plastic sheeting, or web, is provided. Wells are thermoformed in the web. The wells may be formed in an 8 by 7 index. The wells may be filled with flash-sterilized insect diet through a manifold. The diet is then cooled to solidify it. After cooling the diet, insect eggs are delivered to the wells. The eggs may by in a bulking material. The web may then be heat sealed with a top film.

The sealed web may then be loaded on spools and subjected to conditions suitable to incubate the eggs. After incubation, the web may be passed through an airlock to an expression suite B. The web may be spooled from the insectary where the eggs were incubated to the expression suite. The larvae may be inoculated with inoculum. The web may then be reloaded on spools and the loaded spools incubated. The infected insects may then be harvested.

The process utilizes a continuous web. Materials typically utilized for the web include polyvinyl chloride (PVC). The web (or ribbon) may be formed from a variety of materials that permit wells to be formed therein. Materials typically are selected that have sufficiently sturdiness to withstand physical stresses encountered during the process. Other characteristics that may be useful in selecting a material can include resistance to being eaten by the organisms, such as insects, being reared in them. Permeability to fluids may also be important. Typically, materials are utilized that have little or no permeability to fluids. Price may also be a consideration in selecting a material for the film.

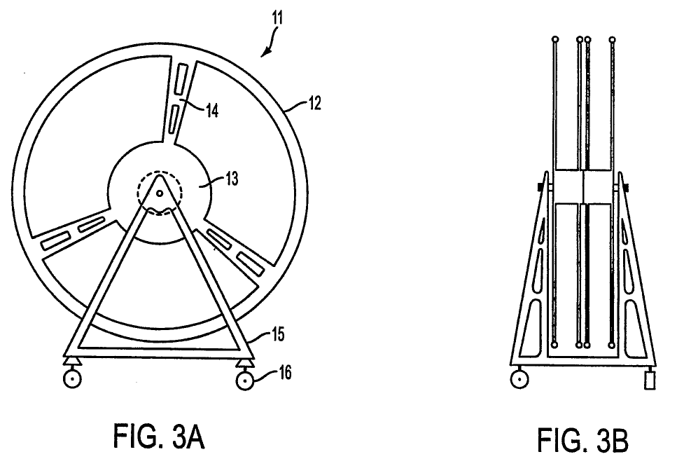

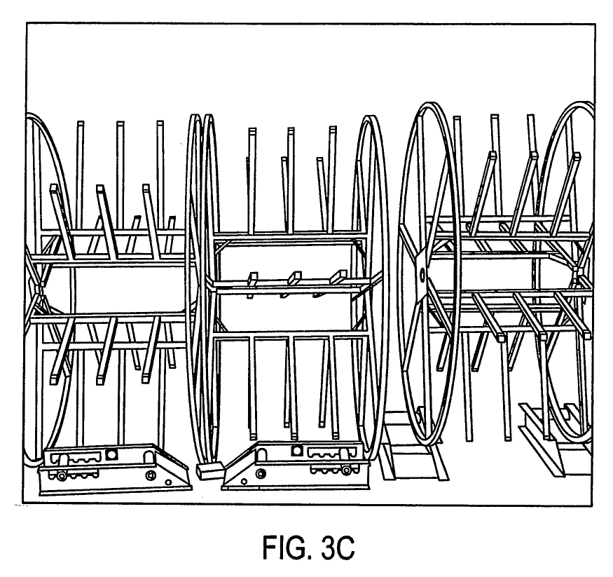

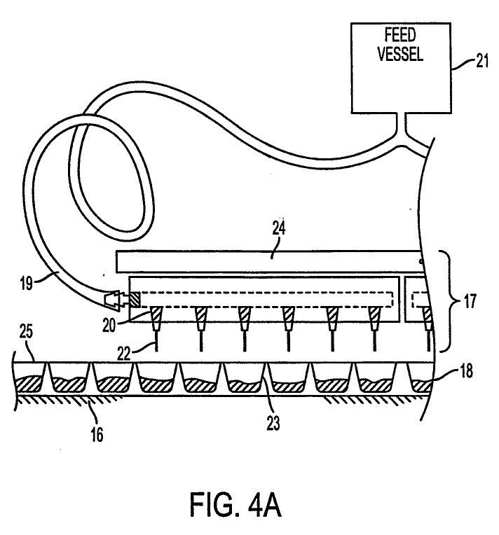

A variety of sizes of web may be employed. Typically, the web, after well formation has a length of 50-800 feet, a width of about 5.875 inches to about 12 inches, and a thickness of about 0.5 to about 4 inches. The higher number may be more typical for a web with large wells to grow multiple individuals per well. The web may have a length of from about 10 feet to several thousand feet and a width from about 5 inches to 5 feet or more. However, after forming, filling and seeding of a wide web, it might have to be sliced to reduce the width of the web when loaded onto the spool. Spacing as provided by the dividers (see FIGS. 3b and 3c) may be necessary to ensure sufficient airflow to each well. According to one exemplary embodiment the web is about 729 feet long, and about 12 inches wide. This web may be split into two ribbons each about 5.875 inches wide. The split improves air circulation as discussed below. Another example embodiment includes a web about 450 feet long. The thickness of the film depends on the type of material, the number and dimensions of the wells and the sturdiness of the web required for spooling the web without compressing the wells. The web may be thinner where the wells formed. One example of a particular web that may be utilized is about 15.0 mil (approx. 375 μm) thick polyvinyl chloride (PVC) film available from Klochner Pentaplast of Rural Retreat, Va.

The web may be provided with wells already formed therein. Alternatively, a matrix of wells may be formed in the web. The dimensions of the wells may vary depending upon the type of organism being raised, the number of individuals in each well, among other factors. Dimensions of the wells may also be important in affecting stability (resistance against compression) to the spooled web. Still other factors that may be taken in to account in determining the dimensions of the wells may include the amount of diet required for rearing the insect; the surface area desired or required for gas and water vapor exchange-the top lidding-sealed surface area is one determinant for gas and water vapor exchange; diet surface exposed to water loss through evaporation, which can be affected by well dimensions; the size of the organism to be reared; and surface covered by inoculum.

According to one preferred embodiment, the wells each have a length of about 1.0625 inch, a width of about 1.0625 inch, and a depth of about 0.75 inch. Each well may include one or more dimples in the bottom of the well. The dimple provides an offset from layers of the web beneath the well, providing channels for an air flow and gas exchange with the wells. According to one particular embodiment, the dimple has a diameter of about 0.5625 inch and a depth of about 0.0625 inch. However, the dimple may have other shapes and/or sizes. The spacing of the wells with respect to adjacent wells and/or the edges of the web may be substantially uniform over the entire web. Alternatively, either or both spacings may vary. For example, where wells are formed in a matrix as described below, the spacing between matrices may be different than the spacing between adjacent wells within each matrix of wells. For example, the spacing could be greater. The distance from the edge of the web to wells at the perimeter of the web may be different than the spacing between adjacent wells. For example, the spacing could be greater.

The wells may be formed according to conventional practices. Typically, wells in a PVC web are thermoformed. The particular process parameters required to form the wells would be known to one of ordinary skill in the art. The wells could be formed in a continuously moving web through similarly moving molding equipment. The wells would be formed as the web and molding equipment move. Alternatively, the web could be held stationary as the wells are formed. In either case, the number of wells formed could vary. For example, one row at a time could be formed. Alternatively a matrix of multiple rows could be formed simultaneously. In such a case, the web could be maintained stationary, the matrix of wells formed and then the web advanced for formation of another matrix of wells.

The number of wells in the web typically varies with the dimensions of the web. In the embodiment described above where the web is about 729 feet long, and 12 about inches wide, the web contains 50,929 wells. A typical array is four wells across. Thus, no well is more than one well away from a side of the web, facilitating gas exchange.

The web may be supplied rolled up with the wells already formed. Alternatively, the web may be supplied rolled up without the wells formed. The web may be unrolled and the wells formed. Then, the web may be rolled up again or the wells loaded with growth medium and eggs.

While the invention may be described in terms of raising insects, it may also be utilized to raise other organisms, such as spiders, or worms, with appropriate adjustments to dimension and feed. The invention may also be utilized to rear any other organism that could be reared in such wells. Examples of insects that could be reared with the system and method according to the present invention include Trichoplusia ni (cabbage looper), Spodoptera exigua (beet armyworm), Spodoptera frugiperda (fall armyworm), Heliothis virescens (tobacco budworm), Helicoverpa zea (bollworm), Plutella xylostella (diamondback moth), Ostrinia nubilalis (European corn borer), Anagrapha falcifera (celery looper), Cydia pomonella (codling moth), Cryptophlebia leucotreta (false codling moth), larval stages of other moth species, butterfly species and other insects for which an artificial, gel-based diet is available, predatory insects, parasitic insects, grasshoppers, crickets, katydids (Orthoptera), cockroaches (Blattodea), mantids, walking sticks, earwigs (Mantodea, Phasmatodea, Dermaptera), thrips (Thysanoptera), true bugs, flower bugs (Heteroptera), aphids, planthoppers, leafhoppers, scale insects (Homoptera), Lacewings (Neuroptera), butterflies, moths (Lepidoptera), beetles (Coleoptera), Flies (Diptera), parasitic wasps, and sawflies (Hymenoptera). In addition, examples of spiders include spiders for which an artificial gel-based diet is available, other species of spiders (Araneae), and mites (Acari).

The composition of the growth medium may vary, depending upon the organism being raised. One example is a wheat-soy insect diet available from Bio-Serv Products, Inc. of Frenchtown, N.J. The diet has the following composition:

| Ingredient | Amount(g/L) |
|---|---|
| Agar | 17 |
| Sucrose | 34 |
| Soy Flour, 50% | 39 |
| Wheat Germ, Stabilized | 34 |
| Wesson Salt Mix | 10 |
| Alphacel | 5 |
| Sorbic Acid | 1 |
| Methyl Paraben | 5 |
| Ascorbic Acid | 4 |
| USDA Vitamin Premix | 12 |

The Wesson Salt Mix included in the diet may have the following composition:

| Ingredient | Amount (%) |
|---|---|
| Calcium carconate | 21 |
| Copper sulfate 5H₂0 | 0.039 |
| Ferric phosphate | 1.47 |
| Manganese sulfate (anhydrous) | 0.02 |
| Magnesium sulfate (anhydrous) | 9 |
| Potassium aluminum sulfate | 0.009 |
| Potassium chloride | 12 |
| Potassium dihydrogen phosphate | 31 |
| Potassium iodide | 0.005 |
| Sodium chloride | 10.5 |
| Sodium fluoride | 0.057 |
| Tricalcium phosphate | 14.9 |

The USDA Vitamin Premix included in the diet may have the following composition:

| Ingredient | Units/kg | |
|---|---|---|
| Vitamin A | 22,000,000 | IU |
| Vitamin E | 7,999 | IU |
| Vitamin B₁₂ | 2.002 | mg |
| Riboflavin | 499 | mg |
| Niacinamide | 1,012 | mg |
| d-Pantothenic Acid | 920 | mg |
| (ca d-Pantothenate) | (1001 | mg) |
| Choline | 43,999 | mg |
| (Choline CI) | (49,999 | mg) |
| Folic Acid | 251 | mg |
| Pyridoxine | 205 | mg |
| (Pyridoxine HCI) | (251 | mg) |
| Thiamine | 222 | mg |
| (ThiamineHCI) | (251 | mg) |
| d-Biotin | 20.2 | mg |
| Inositol | 20,000 | mg |
| Dextrose | (Add to 1 | kg) |

Corn Cob Grit may also be included in the diet. According to one embodiment, 20/40 mesh corn cob grit is utilized. Such corn cob grit is available from The Andersons Company of Maumee, Ohio.

The growth medium and/or insect eggs may be dispensed into the wells utilizing any suitable means. Typically, the growth medium and/or insect eggs may be dispensed utilizing an automated apparatus that permits a number of wells to be loaded simultaneously. According to preferred embodiments, the wells are formed, growth medium loaded in the wells and insect eggs are supplied with a single apparatus.

A number of apparatuses exist for carry out the well formation and loading operations, which may be referred to as form, fill and seal (FFS). One example of such an apparatus is a customized horizontal form, fill, and seal machine, model Compact 320 available from Tiromat, a division of Convenience Food Systems, Inc. located at 8000 North Dallas Parkway, Frisco, Tex. There are many other companies that make such machines, including Mahaffy & Harder Engineering Company, Fairfield, N.J.; Multivac, Inc., Kansas City, Mo.; and Harpak, Inc., Easton, Mass.

A separate apparatus may be utilized to supply the diet for the insects to be provided to the FFS machine. This apparatus may also sterilize the diet prior to supplying it to the FFS machine. One example of a diet supplying apparatus is a Unitherm™ aseptic food processing system available from Waukesha Cherry-Burrell of Louisville, Ky. This system continuously pumps liquid insect diet through a series of Spirotherm™ heat exchangers initially heating the diet to a sterilization temperature of about 285° F. The apparatus holds the diet at this temperature for about 2 seconds then cools the diet to about 106° F. This process based in a short product hold time at the sterilization temperature is often referred to as flash sterilization within the food processing industry. At 106° F., an agar based diet is slightly above the gelling temperature, thereby enabling the diet to be dispensed as a fluid through a pump/manifold assembly on the FFS machine.

The FFS machine typically includes a diet dispensing pump and manifold assembly. The diet dispensing system may include a combination of a filling machine (pump) and a dispensing manifold. The dispensing manifold may be a custom assembly for this application. Precision computer guided machining systems may be utilized to fabricate a plenum plate and distribution plate included in the dispensing manifold. One example of a pump that may be included in the assembly is model DAB-32-2 available from National Instrument Company, Inc. of Baltimore, Md. One example of a manifold that may be utilized is available from Mechanical Development, Inc. of Salem, Va. The dispensing system can enable a precise volume of diet to be evenly distributed into discrete streams delivering a process specified volume to each well.

After loading the wells with diet, the web may be passed through a cooling tunnel. The cooling tunnel may be designed to lower the temperature of agar based insect diet from a dispensing temperature of about 106° F. where it is in a liquid state, to about 90° F. where it is in a solid state. The cool down may take place within a resonance time as fast as about 30 seconds. The cooling of the diet may be accomplished with a number of apparatus. One embodiment utilizes a 60 inch cooling tunnel fed with HEPA filtered 4° C. air, flowing counter current to the flow of the form, fill and seal system, and a custom molded cold plate at a temperature of about 1° C., which places the rearing wells in direct contact with the chilled plate on three sides for maximum surface contact. The calculated BTU load for the cold plate is about 10,050 and about 19,958 for the cooling coils providing the chilled air. The cooling tunnel is capable of self defrosting. The chilling tunnel was incorporated into the form, fill, and seal system by Convenience Food Systems, Inc.

Once the diet is loaded into the wells and put through a cooling process and/or undergone any other necessary process, the insect or other eggs may be deposited in the wells. As with the other aspects of the invention, the egg deposition process may be automated. According to one embodiment, the egg depositor is a custom designed system, integrated into the form fill and seal machine. This custom designed apparatus enables the delivery of a defined quantity of eggs, formulated in a corn cob grit bulking material, to each well. It functions via a pneumatically driven shuttle plate which is controlled by the form, fill and seal CPU from orientation feedback provided by proximity switches on the egg depositor. The shuttle plate receives eggs into bored out orifices from a holding bin containing the egg bulking material mixture, then shifts orientation to align with matching orifices located directly above the wells containing solidified insect diet on the form, fill, and seal machine. The vertical movement of eggs is gravity driven, horizontal movement occurs via the pneumatically driven shuttle plate.

The speed of the process of forming the wells, loading the wells with diet and eggs may depend upon the size of the web, and the apparatus used to fill the wells, among other factors. According to one embodiment, the FFS machine operates routinely at about seven indexes per minute. Each index includes 56 wells useful for a 7×8 array of wells. The FFS machine forms the wells, loads with diet and seeds the wells with insect eggs. Therefore, at seven indexes per minute, these operations generate 7×56=392 wells/minute. This embodiment has also been demonstrated to work at nine indexes per minute.

Once the eggs are deposited in the wells, the wells may be covered for the egg incubation and larvae growth. A number of materials may be utilized for the cover. Typically, the cover material is resistant to insects feeding through it. The cover may also be permeable to gas, including water vapor, but not to the extent that the diet becomes too dry during incubation. The cover also permits gas exchange for the organisms within. That is, oxygen needs to permeate in and carbon dioxide needs to permeate out. The specific permeability may depend at least in part on the relative humidity in the incubation area, the amount of diet, the incubation time and/or the temperature, among other factors. The permeability may be established by experimentation for each insect under each method. According to one exemplary embodiment, the cover includes a perforated polyester film (Mylar film), having a thickness of about 200 gauge or 2.0 ml. Other polyester films and Tyvek may alternatively be used.

The cover may be applied to the web with the wells filled with diet and eggs as a sheet. Typically, the cover material is provided as a roll, wherein the cover is unrolled onto the web and secured to the web. The web may be rolled up after attachment of the cover. The cover may be secured to the web in a variety of ways. For example, adhesive may be applied to the web and/or cover to secure the cover to the web. The cover could also be welded to the web, wherein the cover and/or the web is heated to melt the cover and/or the web. Any means may be utilized that secures the cover to the web and seals the wells. According to one preferred embodiment, the cover includes 27HT1/2PET perforated polyester film, 200 gauge (2.0 mil thickness). The cover is secured to the web with 27HT adhesive, hot melt, which can take place at a temperature of about 105° F., heat seal, dot pattern coating. The adhesive is applied to the web and/or cover with a coating weight of about 22 grams/sq. meter.

The film may have a Gurley porosity in the range of about 3-25 sec./100 cc/sq. in. (Vendor: Oliver Products Company, Grand Rapids, Mich.). Porosity is the measure of how easily air can pass through a sheet of paper. The Gurly test measures the time needed to pass a given volume of air through the sample.

After covering the wells, the web is rolled up. Typically, the web is rolled up utilizing a spool or other structure that can support the web and facilitate its rolling up. A spool operative to act as a platform for the web to be rolled up on and unrolled from may be made of a variety of materials, such as metal, such as steel and aluminum, plastic, wood, composite materials and/or other suitable materials. A spool has dimensions sufficient to securely hold the web and to be accommodated in the physical facilities where the processes are being carried out. For example, the outside dimensions of the spool could be restricted by the height and width of doors to incubator chambers. Other factors that could be important in determining spool dimensions could include biological parameters, such as gas exchange, and strength of the web, including the number of layers that the bottom layer can support without collapsing.

According to one embodiment, the spool is made of 1″ square steel tubing. The steel is finished with Rustoleum hammer coat finish. The spool has the following dimensions:

| | | |
|---|---|---|
| Compartments: | 4 × 6.5″ | 26″ |
| Dividers: | 3 × 1.0″ | 3″ |
| Flange: | 2 × 1.0″ | 2″ |
| Travers: | | 31″ |
| Flange Diameter: | | 58″ |
| Drum: | | 20″ |
| Arbor Hole: | | 3.25″ |

FIGS. 3a and 3b illustrate an embodiment of a spool that may be utilized with the present invention. The embodiment shown in FIGS. 3a and 3b may accommodate two webs on the spool 11, which has a rim 12 having a diameter of about six feet. The hub 13 of the spool has a diameter of about seven inches. The hub is connected to the rim with a number of spokes 14. The width of the spool is about 2.5 feet and the supporting carriage 15 about 3.5 feet. As shown in FIGS. 3a and 3b, the spool may include wheels 16 to facilitate its transportation to various locations to carry out various functions of processes of the present invention. FIG. 3c illustrates a plurality of spools according to another embodiment. The spools shown in FIG. 3c can each accommodate four webs.

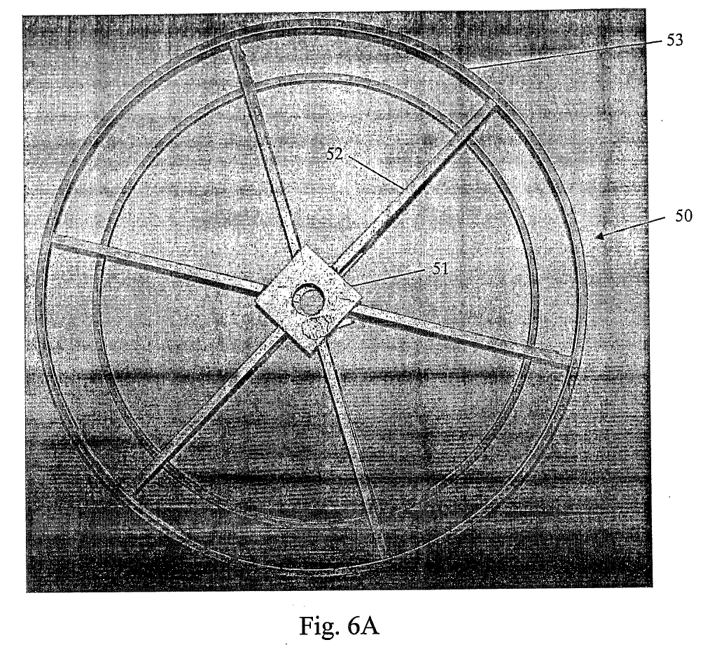

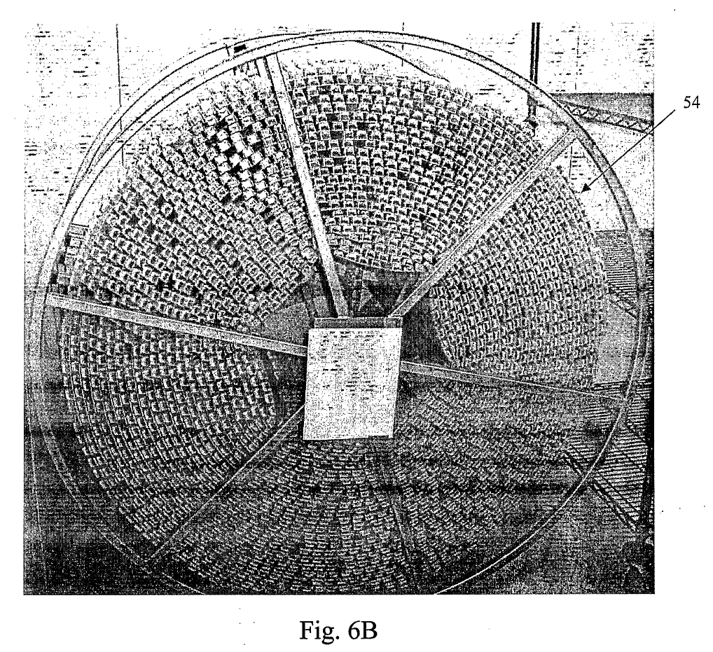

FIG. 6A illustrates another embodiment of a spool 50 that may be utilized with the present invention. The spool 50 shown in FIG. 6 includes a hub 51, spokes 52 and rim 53. FIG. 6B illustrates the embodiment of the spool shown in FIG. 6A with a web 54 rolled up on the spool.

The web may be secured to the spool in a number of ways. For example, the spool could include mechanical clips and/or clamps. The web could also be secured to the spool with tape or another adhesive. According to one embodiment, the web is secured with duct tape or another similarly tacky tape.

After securing the web to the spool, the web is wound up on the spool. The speed with which the web is wound may depend upon the material forming the web, length of web being wound, and/or other factors. The spooling typically requires only a few minutes. The number of windings can depend upon the size of the spool and the length of the web. It may be desirable to wind the web more or less tightly, depending upon the growth requirements of the organisms being raised. Along these lines, the web may be wound more or less tightly to control gas flow to and from the wells and/or to control temperature of the wells, among other things. According to one embodiment, a web about 450 feet long and including about 64,000 wells was wound 49 times on a spool.

The web may be wound on the spool with a motor. The speed, tension and other parameters of the winding may vary, depending upon the particular web material, desired spacing of successive wrappings of the web, among other factors. Typically, the web is wound at low tension. A set of idle rollers may be utilized to provide a passive accumulating loop to accommodate the indexing motion of the FFS machine and the doffing of spools. According to one particular embodiment, the average winder speed is about 27.6 feet per minute utilizing a 1 hp motor.

The take-up of the web on the spool can be manually driven or powered. Manual drive requires a person to roll the spool to wrap the web around the spool. Powered take-up can be accomplished via an independent motor or the drive of the form-fill-and-seal machine can be used. The drive of the machine may maintain the registration of the web. Either way, a powered drive may include a slip drive to keep tension on the web while allowing the web to stop the rotation of the spool. One example of a such an arrangement includes a smooth belt over a polished collar on the spool. The belt drives the spool to rotate and wind the web but if the web stops, due to intermittent motion of the FFS machine or due to a production problem, the belt will slip and allow the spool to wait for the web. The friction of the belt slipping maintains the tension on the web.

After winding of a web on a spool, the eggs may be subject to incubation. The conditions of the incubation depend upon the particular organism being incubated. Those of ordinary skill in the art know the conditions required to incubate insect, spider or other eggs that may be raised according to the present invention.

Having web(s) on one or more spools makes it easy to transport the web to a location for incubation. An incubation location may include racks and/or shelves for receiving one or more spools. Rack and shelves can facilitate placement and removal of the spools. According to one embodiment, spools are arranged on storage racks the accommodate spools one deep and two high. A fully loaded spool with a six foot diameter could weigh on the order of about 400 pounds and could be fully wound in about 14.6 minutes. The spools may be placed on and removed from racks with a narrow aisle reach truck. While the spools may be placed in any orientation for incubation, according to preferred embodiments, the spools are tipped over and arranged such that the spool core axis is vertical. Spools may be stacked six high. Moisture, temperature, air composition, air flow and other parameters may be controlled during the incubation.

After hatching of the eggs and growth of the resulting larvae, the succeeding steps depend at least in part upon the desired end product. As referred to above, the present invention may be utilized to raise mature organisms, to produce virus, or a protein product. If the process is to produce mature organisms, then the larvae may remain in the wells until maturity or removed prior to maturity to mature elsewhere. On the other hand, if the process is for producing virus or a protein product, then the larvae are next inoculated with a virus.

If the present invention is being utilized to raise insects, then the virus could be any suitable insect virus. Along these lines, any baculovirus could be utilized. The virus may be wild-type or a recombinant virus. If the present invention is being utilized for protein production, then a recombinant virus is typically utilized. On the other hand, if the present invention is being utilized for bio-insecticide, either a wild-type or a recombinant virus could be employed.

To inoculate the larvae, the web may be unwound and delivered to an inoculation apparatus. Similar to the apparatus for depositing eggs in the wells, the inoculation may inoculate a plurality of the wells simultaneously. To carry out the inoculation, the web is unrolled and the virus delivered to the wells. The unwinding for this and other operations may be carried out utilizing an unwinding motor. The motor may wind a web from a spool at any suitable rate. According to one particular embodiment, the web is unwound at two feet per minute with a 2 hp motor.

The virus typically is delivered to the wells in an area that is isolated from other areas. This may be accomplished utilizing isolating mechanisms such as an air lock, separate air handling systems, and directional flow of goods and personnel. Air flow mechanisms can include regulating air pressure to maintain air in only one direction. According to one embodiment, an air lock slot between an inoculation area and a pre-inoculation area is utilized. The air lock slot is about 6½″×1″ and is closed when not in use. The airlock maintains airflow from overpressure in an insectary to under pressure in an inoculation area. This helps to limit or prevent danger of contamination of the insectary.

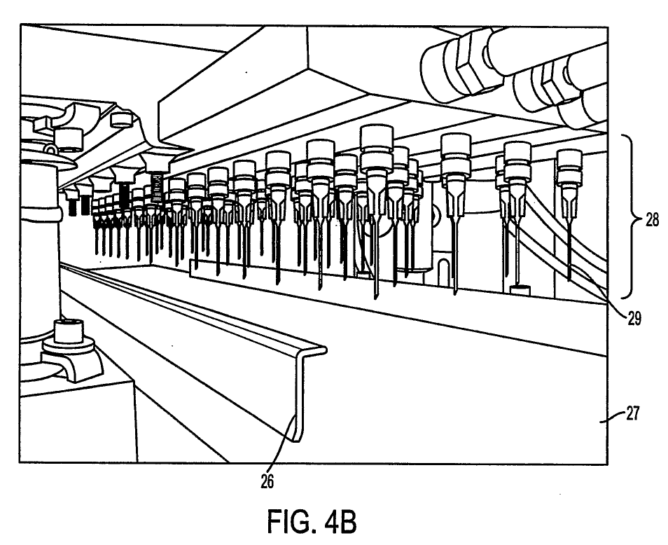

FIGS. 4a and 4b illustrate an embodiment of an inoculation apparatus according to the present invention. This embodiment includes a web supporting surface 16. A virus delivery manifold 17 is arranged over the unrolled web 18. According to one embodiment, the manifold includes an eight leg dispensing manifold 18. For example, the manifold may have a ⅛″ ID× 3/32″ wall of platinum cured silicone tubing available from Mitos Technologies, Inc of Phoenixville, Pa. This embodiment splits one stream 19 of inoculum suspension into eight streams 20, effectively connecting the needle manifold 17 to the feed vessel 21. The embodiment of the manifold shown in FIG. 4a includes 56 needles 22. The needles may be arranged in a needle manifold 17. The needle manifold may hold hypodermic needles 22 that puncture cover material and deliver a discrete dose to the insects housed in the wells 23. Each needle dispenses a preset volume of inoculum into each well. A vertically moving platform 24 to which the manifold 17 is attached lowers the manifold so that the needles 22 penetrate the cover 25 and then the needles dispense a preset volume of inoculum into each well.

A tube 19 interconnects the manifold with a source of virus, such as a feed vessel for holding an inoculum suspension. A pump transports the inoculum suspension from the feed vessel through the needle manifolds, delivering a discrete dose to the insect. One example of a pump is a Brewer Automated Pipeting Machine, model 40A, available from Scientific Equipment Products of Baltimore, Md. Activation of the pump is coordinated with the movement of the platform that moves the manifold assembly.

FIG. 4b illustrates another view of a virus delivery system. The system includes a guide rail 26 to help ensure that the web remains in position such that the wells are aligned with the injection needles. The web is supported by web support surface 27 as it travels through the system. This injection system includes an array 28 of injection needles 29.

The inoculation assembly may also include a pneumatic timing system. The timing system could include a cam actuated via integration with dosing pump. The timing system may control raising and lowering of needle manifolds. The timing system could additionally or alternatively include electronic controls, manual and/or any other controls to regulate the operation of the inoculation.

The present invention represents a significant advantage over known processes, which are carried out manually in batches. Along these lines, known processes typically include an operator taking a tray, such as with 56 wells, from the cart and inserting the tray into an inoculator under a platform into which 56 hypodermic needles are placed. Upon pressing a foot switch the inoculator performs three operations: the platform lowers until the hypodermic needles have just penetrated through the top film, then a precise volume (e.g., 200 microliters) of inoculum is sprayed onto the diet, and then the platform raises. A second operator collects the trays and stacks them onto a cart. The present invention can reduce manual labor by about 80%.

After inoculation the web may be rerolled. As the inoculation takes place, a portion of the web may be rolled, awaiting inoculation, a portion may be unrolled and undergoing inoculation, and a portion may be rolled after being inoculated. Once inoculation is complete, the rolled, inoculated web may be subjected to conditions to incubate the virus. Those of ordinary skill in the art would know suitable conditions for incubating particular viruses.

At a point depending upon the purpose of raising the insects, the larvae may be harvested. Harvesting may take place once the insects infected with the viruses have undergone incubation, or once the larvae reach a desired state of maturity if virus or protein production is not a part of the process. Harvesting may preserve the larvae or sacrifice them again depending upon the purpose of the rearing of the insects.

The harvesting may include shredding the web, cover and contents and the separating the desired product from the shredded mixture. Alternatively, the cover may be peeled from the web, the contents removed from the wells, the larvae separated from the other contents. The larvae could then be processed to isolate protein or virus. Alternatively, the larvae could be raised to release mature insects.

According to one embodiment of a harvesting, in processing a spooled web for harvesting the leading edge of the cover is attached to a cylinder for winding the cover. A motor winds up the cover as the web advances. In fact, the same motor could power both the spool unwinding and the cover removal.

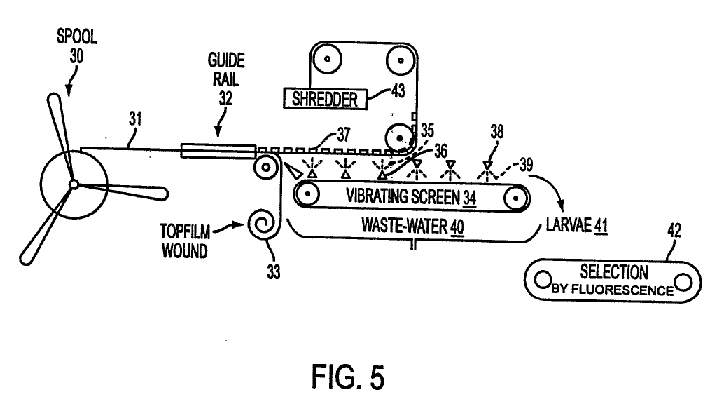

One embodiment of a harvesting apparatus is shown in FIG. 5. In the embodiment shown in FIG. 5, after the web 31 is unrolled from the spool 30, it travels along a guide rail 32. The cover 33 is removed by peeling it off of the web. As the cover is removed, the contents of the web fall on a vibrating screen 34. Fluid 35 may directed by jets 36 at the wells 37 to facilitate removal of the larvae and other contents. Additionally, jets 38 may direct fluid 39 toward the material on the screen to facilitate separation of the larvae from the other contents of the wells. The other contents of the wells typically have smaller sizes than the larvae and so fall through the screen as waste 40. The larvae 41 are then collected 42. The web is sent to a shredder 43.

One embodiment of a harvesting machine includes a vibratory screening machine, available from Royson Engineering Company of Hatboro, Pa., to harvest infected larvae expressing protein from the rearing trays. This embodiment utilizes a seamless type 304 SS single panel screen, 8×8 mesh per sq. inch, wire diameter: 0.0280″, opening size: 0.0970″, open area: 60.2%, oriented at ¼ pitch to carry larvae horizontally down the screen to a drop-off for collection while allowing digested diet and debris to pass through the screen as waste which is collected at a separate drop-off.

The present invention permits automation of aspects of insect rearing. Along these lines, preferred embodiments of the present invention can increase the unit of handling by winding the web on a spool. Handling spools that weigh several hundred pounds instead of separate trays or cuts of less than a pound in weight leads to tremendous gains in efficiency. The size of tray utilized according to known processes is limited by the weight and dimensions that can be comfortably handled by an operator.

In addition to benefits of handling a spool of the web rather than large numbers of individual trays, no attention is typically needed while the web is being wound onto the spool. This can reduce operator time by a minimum of 80%. As a result, by increasing efficiency, the present invention can reduce labor costs.

Handling of the web as opposed to many trays can lead to benefits in the inoculation process as well. According to currently utilized processes, the trays are fed into an inoculator device. This operation is most efficiently done with two operators, one taking the trays off the cart and feeding them into the inoculator while the other takes the inoculated trays and places them onto another cart. Using a spool-based system, only one operator is needed to monitor the operation, replacing two operators. Additionally, the speed of the inoculation is not limited by the speed of the operators feeding the trays into and removing trays from the inoculator. Also, the number of operators may be reduced from two to one and the time to carry out inoculation may be shortened due to higher speed of the inoculator. Operator time may be reduced by an estimated 80%.

The present invention also is particularly adapted to the harvest of larvae for protein production. This process is time critical due to the sensitivity of proteins to degradation. Therefore, larvae need to be harvested prior to death. By more quickly harvesting the larvae, the present invention addresses this issue. Furthermore, in harvesting the larvae, the present invention can achieve an advantage in labor savings over known processes. Along these lines, the present invention can utilize only one operator who monitors the harvester equipment to replace four or more operators necessary to accomplish the same work manually. Operator time is reduced by a minimum of 80%.

We claim:

1. A method for mass rearing of insects, the method comprising:
providing a web including a plurality of wells;
loading insect eggs or larvae and growth medium in the wells;
covering the wells;
rolling up the web;
incubating the eggs or larvae during eggs hatch and larval growth;
unrolling the web; and
harvesting the larvae.

2. The method according to claim 1, wherein rolling up the web comprises:
attaching the web to a spool; and
winding the web on the spool.

3. The method according to claim 1, wherein harvesting the larvae comprises:
uncovering the wells; and
separating the larvae from other portions of the contents of the wells.

4. The method according to claim 1, wherein harvesting the larvae comprises:
shredding the web, cover and contents of the wells;
separating the larvae from other portions of the shredded web, cover and well contents.

5. The method according to claim 1, further comprising prior to harvesting the larvae:
unrolling the web;
inoculating the larvae with a virus;
rerolling up the web; and
incubating the inoculated larvae.

6. The method according to claim 5, further comprising:
isolating the viruses from the larvae.

7. The method according to claim 5, wherein the web is unrolled into a sterile workspace.

8. The method according to claim 5, wherein the virus causes the larvae to produce an exogenous protein, the method further comprising:
isolating the protein from the larvae.

9. The method according to claim 1, further comprising:
raising the larvae to adults.

10. The method according to claim 1, further comprising:
forming the wells in the web.

11. The method according to claim 1, further comprising:
unrolling the web prior to loading the insect and growth medium in the wells.

12. A system for mass rearing of insects, the system comprising:
a web including a plurality of wells operative to receive insect eggs and growth medium; and
a first spool operative to roll up the web after loading the wells with insect eggs and growth medium, to store the web at least during incubation of the eggs and larvae after hatching of the eggs, and to unroll the web to harvest the larvae.

13. The system according to claim 12, further comprising:
a second spool operative to roll up and store the web prior to loading the wells with insect eggs and growth medium and unroll the web for loading the wells with insect eggs and growth medium.

14. The system according to claim 12, further comprising:
a third spool operative to roll up and store the web after inoculation of insect larvae with a virus and to unroll the web to harvest the larvae.

15. The system according to claim 12, further comprising:
a harvester operative to receive the web from the first spool and peel a cover off of the wells.

16. The system according to claim 15, wherein the harvester is operative to remove the contents of the wells and separate insect larvae from other portions of the contents of the wells.

17. The system according to claim 15, wherein the harvester is operative to shred the web and contents of the web and separate insect larvae from other portions of the contents of the web.

18. The system according to claim 12, further comprising:
an inoculator operative to inoculate the insect larvae with a virus.

19. A harvester for harvesting insect larvae from a web of wells, the harvester comprising:
a web unwinder operative to unwind the web from a spool on which it is wound;
a top film remover operative to remove the top film from the web of wells; and
a separator operative to isolate insect larvae removed from the wells.

20. An inoculator for mass inoculation of insect larvae with virus, the inoculator comprising:
a web unwinder operative to unwind a web of wells, each well containing at least one insect larvae;
a web support and guide operative to support and guide the web during inoculation;
a matrix of injection needles operative to inject inoculum into the wells;
an inoculum manifold operative to deliver inoculum to the matrix of needles; and
at least one motor operative to alter the location of at least one of the web and the matrix of injection needles after injecting inoculum in the wells.

21. A structure for mass rearing of insects, the web comprising:
a web having a length of about 10 feet to several thousand feet and a width of about 5 inches to about 5 feet;
a matrix of wells arranged in the web;
growth medium and insect larvae arranged in the wells; and
a cover covering the wells.

22. The structure according to claim 21, further comprising:
a spool on which the structure is wound.

23. A system for mass rearing of insects, the system comprising:
means for forming a plurality of wells in a web of material;
means for depositing diet into each well;
means for depositing at least one insect egg in each well;
means for covering the wells with a cover; and
means for rolling and unrolling the web.

24. The system according to claim 23, further comprising:
means for inoculating the insects with an inoculum.

25. The system according to claim 23, further comprising:
means for incubating the insect eggs.

26. The system according to claim 23, further comprising:
means for harvesting insect larvae.
